(19) United States
(12) Patent Application Publication
(10) Pub. No.: US 2008/0263820 A1
(43) Pub. Date: Oct. 30, 2008

(54) Self-Cleaning Handle

(76) Inventor: Wilhelm Blatz, Bruchkobel (DE)

Correspondence Address: DENNISON, SCHULTZ & MACDONALD, 1727 KING STREET, SUITE 105, ALEXANDRIA, VA, 22314, US

(21) Appl. No.: 11/817,279

(22) PCT Filed: Mar. 2, 2006
(86) PCT No.: PCT/EP06/01912

§ 371 Date: Oct. 29, 2007

(30) Foreign Application Priority Data
Mar. 2, 2005 (DE) 10 2005 010 141.0

(51) Int. Cl. E05B 1/00; A61L 2/00

(52) U.S. Cl. 16/110.1; 422/301

(57) ABSTRACT

A self-cleaning handle, in particular a door handle, door knob or similar, includes a handle surface which can be rotated by a drive in relation to a cleaning unit, to perform a self-cleaning process. To achieve simple construction and operation, the handle surface is configured from a large number of surface segments, which are arranged around the periphery of a cleaning unit, and these surface segments can be rotated about their longitudinal axes.

The invention relates to a self-cleaning handle, particularly a door handle or similar device with a grip surface, which, through a drive that activates a self cleaning device, can be rotated relative to a cleaning device and directly or indirectly interacts with same.

DE 200 00 432 U1 describes a self-cleaning handle of the type described above. The familiar handle is a hygienic door handle with a battery-operated control. It transmits an electric current to an electric motor subsequent to operation of the door handle and therefore activates a gear. This causes the grip surface of the outer casing of the handle to rotate around its own axis and a cleaning arm, which is fastened below the outer casing, thereby disinfecting the grip surface of the handle. Lateral bearings and intermediate supports aid in stabilizing the rotation of the self-cleaning door handle. A threaded outer protective cap stabilizes the handle within. The cleaning arm, which is positioned underneath the outer casing, can possibly obstruct the grasping of the handle.

DE 198 57 268 A1 refers to an object with a surface, which is comprised of a fluid guide that is positioned on the inside of the object as well as a manifold that is positioned underneath the surface of the object and joined to the fluid guide in a fluid connection, which distributes the fluid at least onto a partial area of the surface. The object can be comprised of a door handle, toilet seat or even a floor covering. The object is not designed with a device for wiping off fluids.

DE 213 147 U1 also discloses a hygienic, self-cleaning handle for the opening and closing of locks for doors, windows, drawers and similar devices, whereas the operation of the handle causes the movement of a latch or a switching procedure and where disinfecting agents are linked with the handle. The handle is characterized by the disinfecting agents being comprised of a receptacle for a disinfecting agent, whereas the handle is a moving part that is guided within the receptacle for the disinfecting agent, whereby a first section of the handle is always positioned outside of the receptacle and a second section of the handle is positioned inside of the receptacle and whereby an activation of the handle applies an actuating force to the handle, which moves the contacted section of the handle after it has been released into the receptacle and whereby a disinfected section of the handle emerges from the receptacle.

WO-A-03/050366 describes another embodiment of a self-cleaning handle. It comprises an interior base element that comprises a disinfecting agent. The handle is designed in such a manner that the disinfecting agent is transported from the basic body to the exterior of the handle through one or several handle elements and/or as the result of a rotation of the handle elements. The handle elements can be comprised of a porous material such as ceramic, which allows the handle elements to be saturated. The handle elements can alternatively or additionally be comprised of roller elements, which allows for the rotational movement.

The present invention has the goal of developing a self-cleaning handle of the initially described type in such a manner that an automatic self-cleaning of possibly the entire handle surface can be accomplished with a simple construction and handling can be accomplished without the need for a user to come into contact with the cleaning device.

This goal is achieved by a first embodiment according to the present invention in that the handle surface is comprised of a multitude of surface segments which are positioned to be rotational and which are positioned around the perimeter of the cleaning unit, while the surface segments are able to rotate around their longitudinal axis.

The invention-related solution according to the first embodiment is characterized by the handle surface being comprised of a multitude of surface segments which surround the perimeter of the cleaning unit in their entirety. This is advantageous with respect to the state of technology by the cleaning unit being protected against access by a person which, on the one hand, aids in further improving the hygiene and, on the other hand, avoids the interfering with the cleaning unit when the handle is manipulated. The surface segments are segments of roller elements that are positioned around the perimeter of the cylindrically designed cleaning unit.

An alternative, imaginative embodiment to resolve the problem proposes that the handle comprises an energy storage device which, through manipulation of the handle from its resting position to an end position, can be charged with energy, whereby the handle surface during a return movement of the handle can be rotated into the starting position by means of the drive, which is powered by the stored energy.

A second alternative variant of the invention stands out against the state of the art in that the self-cleaning handle can be applied without an external energy source. The energy, which is stored inside of an energy storage device, for example a return spring that is present inside of a door lock, and created when the door handle is depressed, is preferably used to activate the drive that is integrated in the door handle, which performs a relative movement that causes a self-cleaning between handle surface and/or handle surface segments and the cleaning unit.

The drive is preferably provided with an override clutch, where a rotation of the drive is suppressed by the override, when the handle is activated and when the handle is released, a known spring element will return it to its resting position, whereby the drive performs a rotation by blocking the override clutch during the backward movement of the handle, whereby the handle surface or the handle surface segments, which are coupled with the drive, are rotatable relative to the cleaning unit for self-cleaning.

Another imaginative embodiment solves the problem by the cleaning unit incorporating a cylindrical fluid container which is filled with a fluid or, alternatively, connected with a fluid reservoir, and whose radial outer wall is permeable to fluids and will release fluid upon activation of the handle. A tubular cleaning element, such as a sponge, is positioned coaxially in relation to the fluid container, which abuts with its interior surface the outer wall of the fluid container and the surface segments of the roller elements are abutting at its exterior. The tubular cleaning element is preferably comprised of a material such as Polyethylene (PE) or Polypropylene (PP).

The roller elements are positioned around the circumference of the tubular cleaning element, so that it is largely protected against access from the outside. This also has the advantage that the surface segments of the roller elements that form the handle surface are already dry when they reach the surface following multiple applications.

It is furthermore preferred that, starting at a handle support positioned at one door, bar-shaped connector elements, which support the roller elements, continue in the direction of a free end of the handle that is shaped as a counter bearing, and are connectable with same, that the fluid reservoir with a first frontal face is coupled with the drive that is positioned within the handle support and is locked with a second frontal face in the counter bearing, that the roller elements are positioned to rotate on the bar-shaped connector elements, that the drive with the roller elements and the fluid container are coupled in such a manner that during a rotational movement of the drive in relation to a specified angle the roller elements can be rotated relative to a cleaning element and fluid is simultaneously released from the fluid container.

In order to connect the drive with the fluid container, it is planned that the drive is comprised of a pan-shaped bushing to receive the first frontal face of the fluid container, whereby recesses and/or notches are formed in the frontal face that interact with the cup-shaped bushings which are formed in the bottom area in such a manner that a rotational movement of the drive around its longitudinal axis causes a compression of the fluid container that is locked in the counter bearing, that the outer wall of the fluid container has openings, such as slots, which release fluid to the cleaning element through the axial compression of the fluid container that is caused by the drive.

In order to supply the fluid container with fluid, it is planned that the first frontal face of the fluid container have an opening to connect a supply line that is connected with a fluid reservoir.

The outer wall of the fluid container is preferably comprised of synthetic material such as Polypropylene (PP) or Polyethylene (PE) with a wall thickness in the range of 0.2 mm to 1 mm, preferably of 0.5 mm.

In order to affect the rotational movement of the roller elements, it is planned that an outer edge of the drive interact positively in a locked or fitted manner with the roller elements. The outer edge is preferably comprised of a rubberized surface or a rubber band, which abuts the surface segments of the roller elements positively locked.

The self-cleaning handle is preferably a door handle. The handle retainer, which receives the drive, has an angular shape and is connected at its first end with door hardware and whereby at the second end the handle is fastened, whereby the drive comprises a cardan joint, which is positioned inside of the handle support, and which comprises at a first frontal face the cup-shaped receiver for the fluid container and the rubberized outer edge for the drive of the roller elements, and which comprises at its second frontal face a bevel gear that interacts with a frontal face mounted bevel gear that is installed on a shaft which continues vertically relative to the door level, whereby the shaft extends from an override clutch mechanism that is attached to the door, so that, when the door handle is depressed, the override clutch is initially activated through the cardan joint and, when the door handle is released, subsequently through a known spring mechanism returned to its resting position, whereby the override clutch is blocked and the cardan joint is put into rotation, whereby the roller elements are rotated relative to a cleaning element in a specified angle and the fluid container is compressed to release fluid until the gears that are positioned between the notches and recesses disengage and the fluid container returns to its resting position.

According to a preferred version, the fluid reservoir is integrated into the door hardware.

Additional details, advantages and characteristics of the invention are not only found in the claims, the characteristics found therein—individually and/or in combinations—but also in the following description of one of preferred embodiments contained within the illustrations. Shown are:

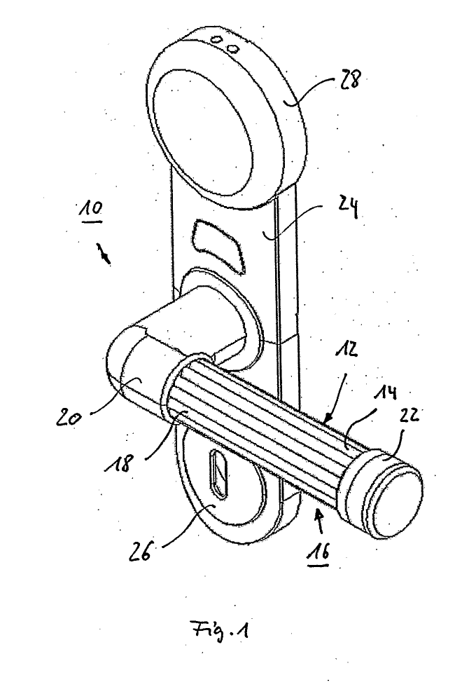

FIG. 1: the self-cleaning handle as door handle in a perspective view,

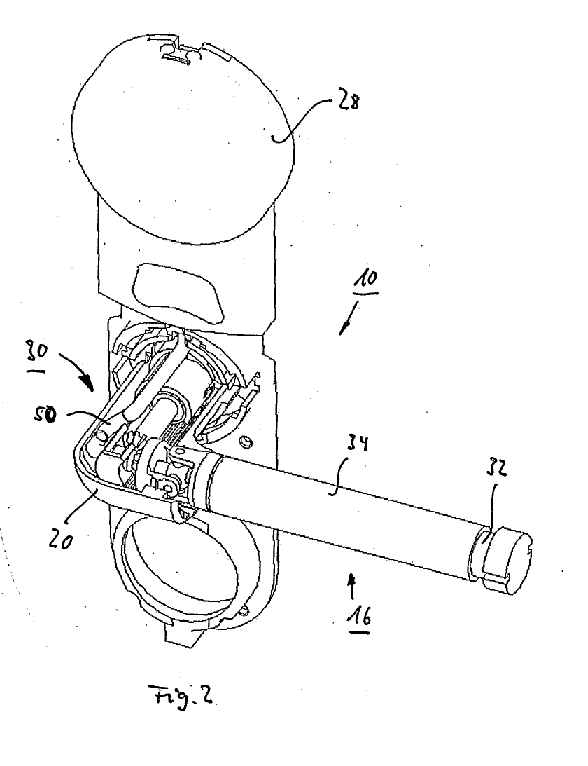

FIG. 2: the door handle according to FIG. 1 in a perspective view, partially cross-sectional,

FIG. 3: a fluid container of the door handle in a perspective view,

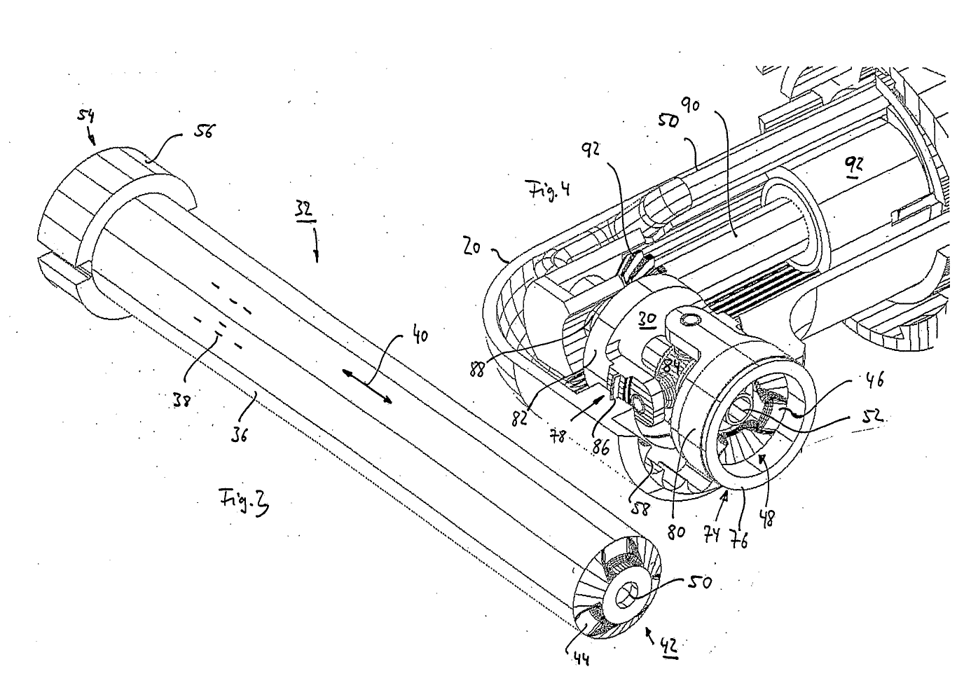

FIG. 4: a drive of the self-cleaning handle in a perspective view,

FIG. 5a-c: a handle support of the self-cleaning handle in a perspective view and in various illustrations,

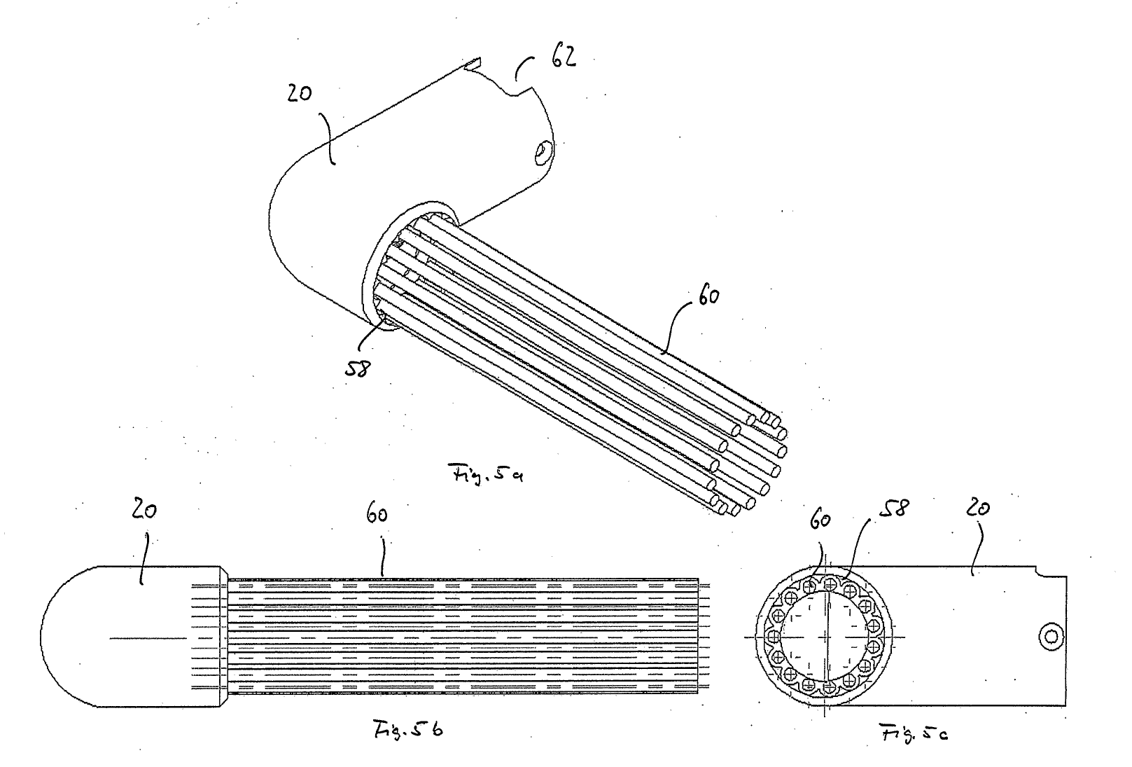

FIGS. 6a and b: a cylindrical arrangement of roller elements in a perspective view and in various illustrations,

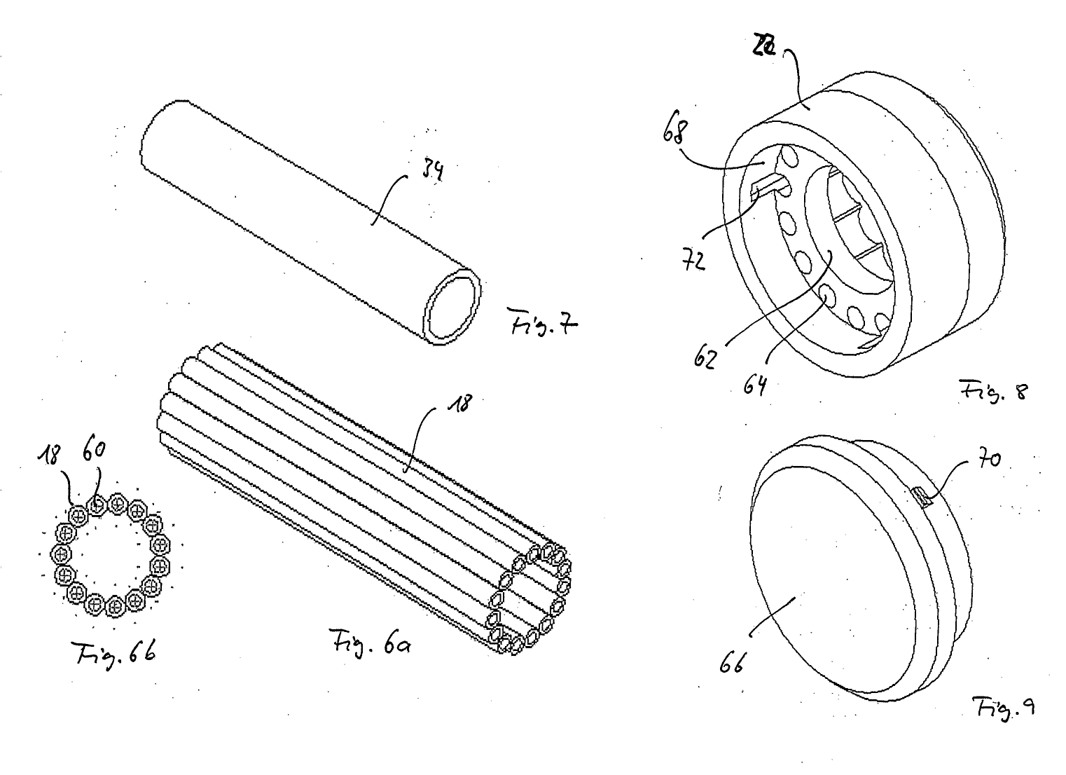

FIG. 7: a tubular sponge element in a perspective view and in various illustrations,

FIG. 8: an end cap of the self-cleaning handle that is formed as a counter bearing in a perspective view,

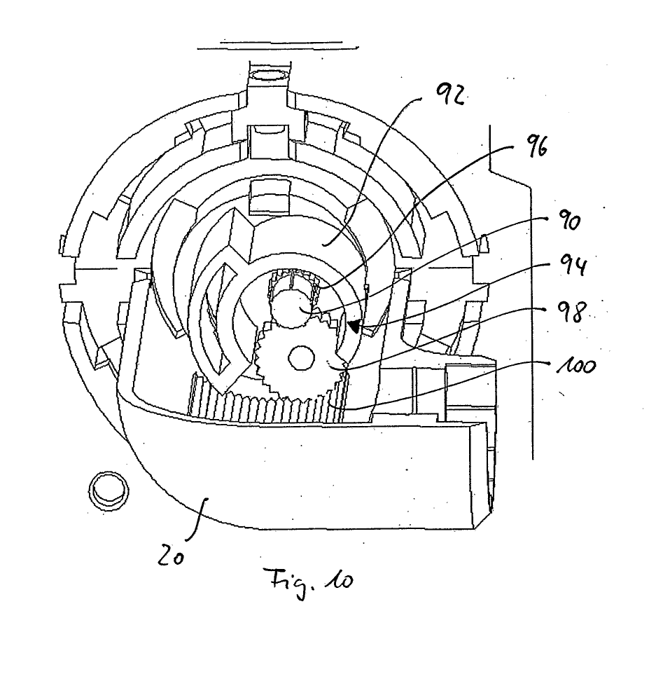

FIG. 9: a cover for the end cap according to FIG. 8 in a perspective view, and

FIG. 10: a detail of the drive of the self-cleaning handle in a perspective view.

FIG. 1 shows a perspective view of a self-cleaning, hygienic handle 10, particularly of a door handle for the interior area of toilet doors, for example in public toilets or in hotels, hospitals, medical offices, bars, restaurants, child care facilities, schools, discotheques, offices and municipal facilities.

The illustrated embodiment shows the handle 10 as a door handle, and it is named as such in the following.

The door handle 10 includes a handle surface 12, which according to the invention is formed of a multitude of surface segments 14, that are formed of roller elements 16 and are peripherally positioned around a cylindrical cleaning unit 16. The roller elements extend thereby between a handle support 20 and a counter bearing 22. The handle mounting 22 has an angular shape and is connected with the door lock (not shown). Also provided is the door hardware 24, 26, whereby the upper door hardware 24 simultaneously accepts a fluid reservoir 28 to supply the cleaning unit 16.

FIG. 2 shows the self-cleaning handle 10 in a partial cross-section illustration, whereby the cleaning unit 16 can be seen, which is connected to a drive 30 housed in a handle support 20. In this context, the cleaning unit 16 is comprised of a fluid container 32 and a tubular cleaning element 34 which continues coaxially in relation to the fluid container, such as a sponge.

The fluid container 32 is shown in detail in FIG. 3. It has a cylindrical outer wall 36, preferably made from synthetic material such as PE or PP with a preferred strength of 0.5 mm. Preferably longitudinally extending slots 38 are installed into the outer wall 36 that, when the fluid container 32 is compressed, will release fluid, which preferably is slightly pressurized and is contained in the fluid container, into the direction of arrow 40 and therefore onto the exterior tubular sponge 34.

The fluid container is provided on its first frontal face 42 with recesses 44 which engage into protrusions 46 in a cup-shaped receiver 48 of the drive 30 shown in FIG. 4. Additionally, the frontal face 42 is comprised of an opening 50 to connect a feeder line 52 for the purpose of connecting the fluid container 32 with the fluid reservoir 28. On a second frontal face 54, the fluid container 32 is equipped with a locking ring 56 which engages with the counter bearing 22, and is locked there.

In order to mount the roller elements 18, bar-shaped connector elements 60, as illustrated in FIGS. 5a-5c, extend from the frontal face 58 of the handle support 20 that are firmly connected with the handle support 20 and delimit a cylindrical sleeve. Another end 62 of the handle support 20 is fastened to the door lock (not shown). The roller elements 18 shown in FIGS. 6a and 6b can be slipped onto the bar-shaped connector elements 60, so that the roller elements can be rotated around their longitudinal axis. The surfaces of the roller elements are thus making contact to the extent that a relative movement of the roller elements 18 towards one another is possible without suffering any friction losses.

The tubular cleaning element 34 shown in FIG. 7 is preferably formed from a material such as Polyethylene (PE) or Polypropylene (PP). The cleaning element 34 is comprised of an inner diameter in such a way that it is adjusted to the outer diameter of the fluid container 32 and is adjusted to the inner diameter of the circular arrangement of the roller elements 18 according to FIG. 7b.

The counter bearing 22, which, on one side, receives the bar-shaped connector elements 60 and, on the other side, the locking ring of the fluid container 32, is illustrated in FIG. 8. The counter bearing is formed cylindrically with a center ring 62, into which drilled holes 64 are made to receive ends of the bar-shaped connector elements 60. The ends of the connector elements 60 are firmly connected in the drilled holes 64, such as being glued or welded. A cover 66 is additionally provided according to FIG. 9, which closes off a frontal opening 68 of the counter bearing 22. At the edge, ratcheting elements 70 are provided that engage into the recesses 72 of the counter bearing.

In the following, the drive 30 shall be described further in respect to FIG. 4. As previously mentioned, the fluid container 32 with its frontal face 42 is positioned in the cup-shaped receiver 48 of the drive 30. The tubular cleaning element 34, which has a diameter roughly equal to the diameter of the drive frontal face 74, extends coaxially to the fluid container 32. Starting at the outer edge 58 of the handle mounting 20, the bar-shaped connector elements continue to where the roller elements 18 are mounted. These in turn are in close contact with the surface of the cleaning element and simultaneously interact positively in a locked or fitted manner with the outer edge 76 of the drive 30. This is according to the here-described embodiment formed as a rubber band or is comprised of same, which during a rotational movement of the drive 30 affects a rotational movement of the roller elements 18.

The rotational movement of the drive 30 occurs at a defined angle within the range of 45° to 90°, so that only surface segments of the roller elements 18 are moving during a rotational movement relative to the cleaning element.

A compression of the fluid container 32 takes place simultaneous with the rotational movement of the drive, whereby the slots 38 that are positioned in the surface 36 are opened, so that the fluid contained in the fluid container is released and the cleaning element 34 is provided with fluid.

The fluid container 32 in its basic position is positioned in the drive in such a manner that the recesses 44 of the frontal face receive the protrusions 46 which are positioned in the drive. A rotation of the drive 30 causes a relative movement between the protrusion 46 and the recesses 44, whereby the protrusions disengage from the recesses during a simultaneous compression of the fluid container 32. After a specified angular rotation of the drive, the fluid container 32 will again torque back into its resting position, whereby the slot-shaped openings 38 are again closed and interrupt the flow of fluids.

According to the execution model of the invention, the drive 30 is comprised of a cardan joint 78 with a first joint section 80, which according to the previous description is connected with the fluid container 32 and the cup-shaped receiver 48 as well as the rubber band 76. A second joint segment 82 is furthermore provided, which is connected through a universal joint 84 with the first joint segment 80.

The second joint segment 82 is rotationally mounted in the handle support 20 by a bearing 86 and has on its frontal face a bevel gear 88. This bevel gear interacts with a bevel gear 92 that is positioned on the frontal face of a shaft 90, whereby the shaft 90 continues vertically or mainly vertically in relation to a level that is formed by the door and ends in an override clutch mechanism 92. The override clutch mechanism according to FIG. 10 comprises a gear 94, consisting of a first gear wheel 96, which is connected to a shaft 90, that interacts with a second gear wheel 98, which engages into a gearing 100 that is formed on the interior side of the handle support 20.

The drive 30 according to execution model shown here functions as follows: The depressing of the handle 10 releases the override clutch, thereby preventing a rotation of the cardan joint 78. The releasing of the handle causes a spring (not shown) to return the handle to its resting position, where the override clutch 92 blocks, so that the shaft 90 and its attached bevel gear 92 remain rigid and will therefore not perform a rotational movement. The upward movement of the handle puts the cardan joint 68 into rotation, which on one side causes the roller elements 18 that abut the rubber band 76 to be rotated at a specified angle and the fluid container 32 to be compressed according to the previous description, in order to release fluid to the cleaning element 34. As soon as the handle has reached its resting position, the rotational movement is complete, the roller elements 18 have been advanced by a specified angular amount and the cleaning element has been supplied with the fluid.

1. Self-cleaning handle (10), particularly a door handle or similar device with a handle surface (12), which for the purpose of self-cleaning can be rotated by a drive (30) relative to a cleaning unit (16) and directly or indirectly interacts with same, characterized by the handle surface (12) being comprised of a multitude of rotationally positioned surface segments which are positioned circumferentially around the cleaning unit (34), whereby the surface segments can be rotated around their longitudinal axis.

2. Self-cleaning handle (10), particularly door handle or similar device with a handle surface (12), which for the purpose of self-cleaning can be rotated by a drive (30) relative to a cleaning unit (16) and directly or indirectly interacts with same, characterized by the handle (10) having an energy storage device which is charged when the handle is manipulated from a starting position into an end position and that the handle surface during the return movement of the handle into the starting position is rotatable by means of drive (30), which is operated by the stored energy.

3. Self-cleaning handle (10), particularly door handle or similar device with a handle surface (12), which for the purpose of self-cleaning can be rotated by a drive (30) relative to a cleaning unit (16) and directly or indirectly interacts with same, characterized by the cleaning unit (16) being comprised of a fluid container (32) which is either filled with fluid or connected with a fluid reservoir (28) and which radial outer wall is formed to be permeable to fluids, and that an activation of the handle (10) causes a release of fluid.

4. Self-cleaning handle according to claim 1, characterized by the surface segments being segments of roller elements (18) which are positioned peripherally around the cylindrically formed cleaning unit (34).

5. Self-cleaning handle according to claim 1, characterized by the drive (30) being comprised of an override clutch (92), where the activation of the handle (10) causes the override clutch (92) to suppress a rotational movement of the drive (30) and where the releasing of the handle (10) causes the handle to be returned to its starting position by the energy storage device, which is formed as a spring element, where the drive by blocking of the override clutch during the return movement of the handle (10) performs a rotational movement, whereby the handle surface (12) or the surface segments, which are coupled with the drive, can be rotated for the self-cleaning relative to the cleaning unit (16).

6. Self-cleaning handle according to claim 1, characterized by the cleaning unit (16) being comprised of a fluid container (32) that is either filled with fluid or connected with a fluid reservoir (28), which radial outer wall is formed to be permeable to fluids and that an activation of the handle (10) causes a release of fluid.

7. Self-cleaning handle according to claim 3, characterized by a tubular cleaning element (34), such as a sponge, being positioned coaxially to the fluid container (32), which with its interior side abuts against the outer wall (36) of the fluid container (32) and the surface segments of the roller elements (18) abut against its exterior.

8. Self-cleaning handle according to claim 1, characterized by bar-shaped connector elements (60) for the support of the roller elements (18) originating from a handle support (20) that is installed at the door and extending in the direction of free ends of the handle (10), which are formed as counter bearings, and which can be connected with same, by the fluid container (32) being coupled with a first frontal face (42) with the drive (30) that is positioned in the handle support (20) and is locked with a second frontal face (54) in the counter bearing (22), by the roller elements (18) being mounted rotatable on the bar-shaped connector elements (60), by the drive (30) with the roller elements (18) and the fluid container (30) being coupled in such a manner that when the drive is rotated in a specified angle, the roller elements (18) are rotated in a specified angle relative to the cleaning element (16) and fluid is simultaneously released from the fluid container (32).

9. Self-cleaning handle according to claim 1, characterized by the drive (30) being comprised of a cup-shaped bushing to receive the first frontal face (42) of the fluid container (32), where in the frontal face (42) recesses and/or protrusions (44) are let in or formed which interact with recesses and/or protrusions (46) which are let in or formed in the bottom area of the cup-shaped bushing (48), in such a manner that a rotational movement of the drive around its longitudinal axis causes a compression of the fluid container (32), which is locked in the counter bearing (22), that the outer wall (36) of the fluid container (32) has openings (38) like slots, which through the axial compression of the fluid container, which is caused by the drive, releases fluid onto the cleaning unit (34).

10. Self-cleaning handle according to claim 1, characterized by the frontal face (42) of the fluid container (32) manifesting an opening (50) to connect a feeder line (52) that is connected with a fluid reservoir (28).

11. Self-cleaning handle according to claim 1, characterized by the outer wall (36) of the fluid container (32) being preferably formed from synthetic material such as Polypropylene (PP) or Polyethylene (PE) with a wall strength in the area of 0.2 mm to 1 mm, preferably of 0.5 mm.

12. Self-cleaning handle according to claim 1, characterized by an outer edge of the drive interacting positively in a locked or fitted manner with the roller elements (18).

13. Self-cleaning handle according to claim 1, characterized by an outer edge (76) being comprised of a rubberized surface or a rubber band which abuts against the positively locked surface segments of the roller elements (18).

14. Self-cleaning handle according to claim 1, characterized by the handle (10) being a door handle, where the handle support (20) that receives the drive (30) is angularly formed and connected with a first end of a door hardware of a door and the handle is mounted to a second end, by the drive (30) comprising a cardan joint (78) that is mounted rotational in the handle mounting (20), and which comprises at a first frontal face the cup-shaped receiver (48) for the fluid container (32) and the rubberized outer edge (76) for the drive of the roller elements (18) and which comprises at its second frontal face a bevel gear (88), which interacts with a frontal face mounted bevel gear (92) that is installed on a shaft (90) that continues vertically relative to the door level, by the shaft extending from an override clutch mechanism (92) that is attached to the door, so that, when the door handle is depressed, the override clutch is initially activated through the cardan joint (78) and, when the door handle is released, subsequently is returned by a known spring mechanism to its resting position, whereby the override clutch is blocked and the cardan joint (78) is put into rotation, that the roller elements (18) are rotated relative to a cleaning element in a specified angle and the fluid container (32) is compressed to release fluid until the gears between notches and recesses disengage and the fluid container returns to its resting position.
